(19) United States
(12) Patent Application Publication
(10) Pub. No.: US 2019/0336396 A1
(43) Pub. Date: Nov. 7, 2019

(54) TABLET PRODUCTION DEVICE, TABLET PRODUCTION METHOD, AND TABLET

(71) Applicant: Freund Corporation, Tokyo (JP)

(72) Inventors: Kiyoshi IMAI, Tokyo (JP); Toshiaki HONDA, Tokyo (JP); Kazuhisa TAKEDA, Tokyo (JP); Eiichi HACHIYA, Tokyo (JP)

(21) Appl. No.: 16/475,169

(22) PCT Filed: Jan. 12, 2018
(86) PCT No.: PCT/JP2018/000649

§ 371 Date: Jul. 1, 2019

(30) Foreign Application Priority Data
Jan. 17, 2017 (JP) 2017-005611

(51) Int. Cl. A61J 3/06; A61K 9/20

(52) U.S. Cl. CPC A61J 3/06; A61K 9/2893; A61K 9/2072

(57) ABSTRACT

A tablet has a hole that can retain an active pharmaceutical ingredient (API) in a side surface. A tablet production device has a second conveyance disk that conveys the tablet while sucking/supporting the side surface of the tablet, medicinal agent supply device ejects the API into the holes and an inkjet head that prints on the front and back surfaces of the tablets. The tablets are conveyed with the side surface thereof sucked by the second conveyance disk, the medicinal agent supply device adds the API into the holes and the inkjet head prints on the front and back surfaces at the same time.

### TECHNICAL FIELD

The present invention relates to a tablet production device that adds active pharmaceutical ingredients (API) to a tablet and, more particularly, to a tablet production device and method that can add API to the side surface of the tablet, and a tablet.

### BACKGROUND ART

Conventionally, a tablet used as medicine is produced by compression-molding (tabletting) a material obtained by appropriately mixing API and an excipient and thereafter applying coating etc. as needed to the molded raw tablet. In recent years, as disclosed in Patent Document 1, there is known a tablet in which API is applied to the surface thereof. Further, as disclosed in Patent Document 2, there is known a carrier tablet in which a concave is formed in the front and back surfaces thereof, and the API is added there.

### CITATION LIST

### Patent Document

Patent Document 1: Japanese Patent No. 4,917,034

Patent Document 2: Japanese Patent No. 5,690,662

### DISCLOSURE OF THE INVENTION

### Problems to be Solved by the Invention

A product number, a product name, a trademark or the like is written on the surface of the tablet for product identification and prevention of accidental ingestion. Such a display on the tablet is made by engraving at tabletting or printing such as transfer printing or inkjet printing. However, in the case of the above-mentioned tablet in which the API is added to the front and back surfaces thereof, printing cannot be applied to the front and back surfaces until the API dries and hence solidifies. Therefore, it is necessary for printing to wait for the API to be dry and this degrades tablet productivity. Further, the API adheres on the front and back surfaces of the produced tablet, so that the API part may peel off the tablet body due to contact with a container or another tablet during conveyance.

### Means for Solving the Problems

A tablet production device according to the present invention includes a conveyance unit that conveys a tablet while sucking/supporting the tablet, wherein the tablet has a concave in its side surface, and the tablet production device has a supply unit that can add predetermined droplets to the concave of the tablet being conveyed by the conveyance unit.

In the present invention, a tablet having, in its side surface, a concave to which predetermined droplets can be added is produced in advance, and the droplets are supplied to the concave. Thus, by changing the type of the droplets to be supplied, different types of tablets can be produced in a single tablet production device. In the device according to the present invention, a significant facility revision is not required for production of various types of tablets, making it possible to reduce device cost. Further, in the tablet produced in the device according to the present invention, the droplets are stored in the concave formed in the side surface of the tablet, making it difficult for the droplet part to contact another tablet during conveyance, which reduces a risk that the droplet added part peels off. The viscosity of the droplets does not matter.

In the tablet production device, the concave may be a hole opened to the side surface of the tablet and extending in the radial direction of the tablet, or a plurality of the concaves may be formed in the circumferential direction of the tablet. In this case, the size of the concave does not matter and may be minute. Further, the droplets may contain a predetermined active pharmaceutical ingredient (API). By forming the plurality of concaves, it is possible to add a plurality of different types of APIs. For example, a combination of APIs in which degeneration is likely to occur due to blending and direct contact between them or a combination of the API and medicinal agents such as an excipient, an absorption promoter, a gel former, and a disintegrator can be added. Further, by incorporating different types of APIs in the plurality of concaves, it is possible to combine tablet drugs for a specific disease in one tablet, which in turn allows production of a tailor-made tablet for each individual patient. As a result, it is sufficient for a patient to take one tablet instead of a plurality of tablets, whereby medication burden can be reduced. Further, the reduction in the number of tablets reduces transportation cost.

The tablet production device may further include a print unit provided in proximity to the conveyance unit and configured to apply printing to at least one of the front and back surfaces of the tablet being conveyed by the conveyance unit.

In the tablet production device, the droplets are added to the tablet side surface, so that normal printing can be applied to the front and back surfaces of the tablet, and the printing onto the front and back surfaces can be performed without waiting for the droplets to be dry. Further, by performing printing as well as addition of the droplets, information corresponding to the type of the droplets can be printed, ensuring traceability of the tablet.

Further, the conveyance unit may suck/support the side surface of the tablet such that the front and back surfaces of the tablet are each fully exposed. Further, the conveyance unit may be a conveyance disk formed in a disk shape, and the conveyance disk may have suction parts arranged in an end face thereof along the circumferential direction and configured to suck the side surface of the tablet.

Another tablet production device according to the present invention includes a conveyance unit that conveys a tablet while sucking/supporting the tablet, wherein the tablet production device has a drilling unit that forms a concave in a side surface of the tablet and a supply unit that can add predetermined droplets to the concave of the tablet being conveyed by the conveyance unit. With this configuration, it is possible to achieve both formation of the concave in the tablet side surface and addition of the droplets in a single device, enhancing versatility of the device.

A tablet production method according to the present invention includes conveying a tablet having a concave in its side surface while sucking/supporting the tablet, and adding predetermined droplets to the concave of the tablet being conveyed.

In the present invention, a tablet having, in its side surface, a concave to which predetermined droplets can be added is produced in advance, and the droplets are supplied to the concave. Thus, by changing the type of the droplets to be supplied, different types of tablets can be produced easily. According to the manufacturing method of the present invention, a significant facility revision is not required for production of various types of tablets, making it possible to reduce device cost. Further, in the tablet produced by the method according to the present invention, the droplets are stored in the concave formed in the side surface of the tablet, preventing the droplet part from contacting another tablet during conveyance, which reduces a risk that the droplet added part peels off.

In the tablet production method, the droplets may contain a predetermined API. Further, the tablet may be conveyed with the side surface thereof sucked/supported, and printing may be applied to at least one of the front and back surfaces of the tablet being conveyed. In the tablet production method, the droplets are added to the tablet side surface, so that normal printing can be applied to the front and back surfaces of the tablet, and the printing onto the front and back surfaces can be performed without waiting for the droplets to be dry. Further, by performing printing as well as addition of the droplets, information corresponding to the type of the droplets can be printed, ensuring traceability of the tablet.

A tablet according to the present invention is a tablet having a hole opened to its side surface and extending in the radial direction thereof, wherein a plurality of the holes are formed so as to be arranged in the circumferential direction of the tablet, and a predetermined API is retained in at least one of the holes.

In the present invention, by forming the plurality of holes, a plurality of types of tablet constituent components, i.e., APIs and various medicinal agents such as an excipient, an absorption promoter, a gel former, and a disintegrator can be added to the tablet. As a result, for example, a combination of APIs in which degeneration is likely to occur due to blending or a combination of the API and medicinal agent such as an excipient can be added.

In the above tablet, different types of APIs may be retained in the holes. As a result, different types of APIs can be distributed into the plurality of holes, so that it is possible to combine tablet drugs for a specific disease into one tablet, which in turn allows production of a tailor-made tablet for each individual patient. For example, by blending enteric granules with an excipient in advance and adding an API that is absorbed by stomach to droplets to be added to the tablet, it is possible to make both the gastro-soluble API and enteric API into one tablet. Further, when a tailor-made tablet is to be produced, it is possible to easily change the amount of the API by changing the amount of droplets for each individual patient, thereby facilitating separate preparations of tablets directed to adults and children, for example. Further, it is sufficient for a patient to take one tablet instead of a plurality of tablets, whereby medication burden can be reduced. Further, the reduction in the number of tablets reduces transportation cost. In addition, by printing a patient's name, a dosing date/time, or a barcode or a two-dimensional code on the tablet itself, medication management can be facilitated. Further, by performing printing in association with past medication troubles due to allergy or DNA data, advanced medication management can be achieved.

A tablet traceability method according to the present invention uses a tablet that has a hole opened to its side surface and extending in the radial direction thereof and in which a predetermined API is retained, and prints information corresponding to the API onto at least one of the front and back surfaces of the tablet so as to allow traceability of the tablet to be ensured.

A tablet medication management method according to the present invention uses a tablet that has a hole opened to its side surface and extending in the radial direction thereof and in which a predetermined API is retained, and prints information corresponding to the API onto at least one of the front and back surfaces of the tablet so as to allow tablet medication to be managed.

### Advantageous Effects of the Invention

A tablet production device according to the present invention having a conveyance unit that conveys a tablet while sucking/supporting the tablet is provided with a supply unit that can adds predetermined droplets to the concave of the tablet having a concave in its side surface, so that by changing the type of the droplets to be supplied, different types of tablets can be produced. Further, in the tablet produced in the device according to the present invention, the droplets are stored in the concave formed in the side surface of the tablet, preventing the droplet part from contacting another tablet during conveyance, which reduces a risk that the droplet added part peels off.

A tablet production method according to the present invention includes conveying a tablet having a concave in its side surface while sucking/supporting the tablet, and adding predetermined droplets to the concave of the tablet being conveyed, so that by changing the type of the droplets to be supplied, different types of tablets can be produced. Further, in the tablet produced by the method according to the present invention, the droplets are stored in the concave formed in the side surface of the tablet, making it difficult for the droplet part to contact another tablet during conveyance, which reduces a risk that the droplet added part peels off.

A tablet according to the present invention is a tablet having, in its side surface, a plurality of holes extending in the radial direction thereof, in which a predetermined API is retained, so that a plurality of types of tablet constituent components, i.e., APIs and various medicinal agents such as an excipient, an absorption promoter, a gel former, and a disintegrator can be added to one tablet. As a result, for example, a combination of APIs in which degeneration is likely to occur due to blending or a combination of the API and a medicinal agent such as an excipient can be added.

A tablet traceability method according to the present invention uses a tablet that has a hole opened to its side surface and extending in the radial direction thereof and in which a predetermined API is retained, and prints information corresponding to the API in the hole onto at least one of the front and back surfaces of the tablet, so that traceability of the tablet can be ensured.

A tablet medication management method according to the present invention uses a tablet that has a hole opened to its side surface and extending in the radial direction thereof and in which a predetermined API is retained, and prints information corresponding to the API in the hole onto at least one of the front and back surfaces of the tablet, so that tablet medication can be managed.

### BRIEF DESCRIPTION OF THE DRAWINGS

FIG. 1 An explanatory view illustrating the configuration of a tablet production device according to a first embodiment of the present invention.

FIG. 2 An n explanatory view illustrating the configuration of a tablet which is one embodiment of the present invention.

FIG. 3 Explanatory views illustrating modifications of the tablet.

FIG. 4 An explanatory view illustrating the configuration of a tablet production device according to a second embodiment of the present invention.

### BEST MODE FOR CARRYING OUT THE INVENTION

### First Embodiment

Hereinafter, embodiments of the present invention will be described. An object of the present embodiment is to provide a tablet production device and method and a tablet that can execute a printing process immediately after addition of API without waiting until the API gets dry. Another object of the present invention is to provide a tablet production device and method that can produce a tablet having a low possibility that the API peels away therefrom and a tablet. Still another object of the present invention is to provide a tablet in which traceability of the added API can be ensured.

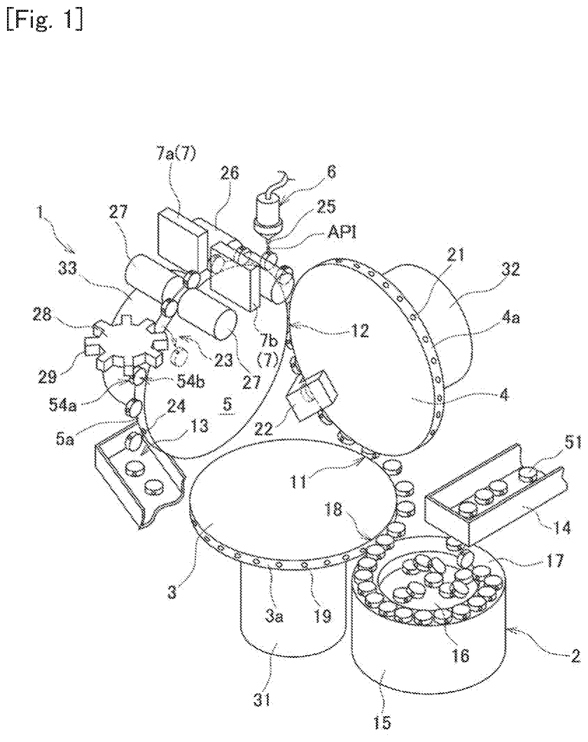

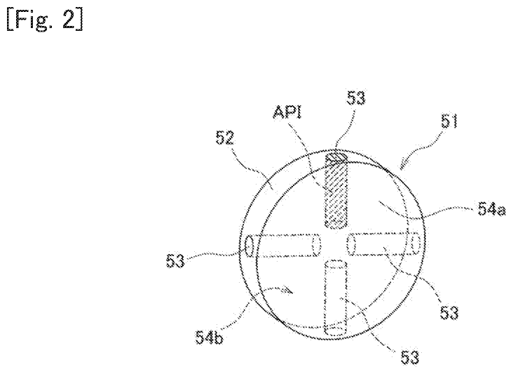

FIG. 1 is an explanatory view illustrating the configuration of a tablet production device 1 according to the first embodiment of the present invention. FIG. 2 is an explanatory view illustrating the configuration of a tablet 51 which is one embodiment of the present invention. A tablet production method according to the present invention is performed in the tablet production device 1 illustrated in FIG. 1, and the tablet 51 according to the present invention is produced in the tablet production device 1. As illustrated in FIG. 2, the tablet 51 has holes 53 in a side surface 52 thereof, into which droplets can be put. The tablet production device 1 ejects the active pharmaceutical ingredient (API) to the hole 53 of the tablet 51 and can thereby produce various tablets having different medical ingredients on a same device. The tablet production device 1 is capable of printing the front and back surfaces of the tablet as well as adding the API. Performing both the printing and API addition makes it possible to ensure traceability and manage the taking of the tablet.

As illustrated in FIG. 2, the hole (concave) 53 that can retain therein the API that is applied thereinto is formed in the side surface 52 of the tablet 51. The hole 53 is drilled along the radial direction of the tablet 51 and opened to the side surface 52. In this example, four holes 53 are equally arranged in the circumferential direction. The innermost part of each hole 53 is closed (dead end), and thus the four holes 53 do not communicate with one another. The holes 53 may be die formed during a molding of the tablet or formed by past-processing after molding. As the tablet 51, a so-called placebo tablet obtained by molding an excipient such as lactose or glucose into a tablet shape is used. The tablet production device 1 is configured to perform both addition of the APIs to the holes 53 and printing onto the front and back surfaces 54a and 54b of the tablet 51.

The tablet production device 1 sucks and conveys the tablets 51 fed by a rotary feeder 2 one by one by three disk-shaped conveyance units (tablet supply disk 3, and first and second conveyance disks 4 and 5) and performs both addition of the API and printing on a single device. The tablet supply disk 3, and first and second conveyance disks 4 and 5 are arranged such that rotary shafts 31, 32, and 33 thereof are perpendicular to one another. The disks 3, 4, and 5 are synchronously driven so that the tablet 51 is conveyed at the same speed. In the tablet production device 1, there are provided a medicinal agent supply device (supply unit) 6 that performs addition of the API and an inkjet head 7 (print unit) that applies desired printing to the front and back surfaces (surfaces to be printed) 54a and 54b of the tablet 51. In the tablet production device 1, the medicinal agent supply device 6 and inkjet head 7 are disposed so as to face the second conveyance disk 5. The tablet 51 is conveyed on the second conveyance disk 5 and subjected to addition of the API and printing thereon.

In the tablet production device 1, the tablet 51 is first sucked and conveyed by the tablet supply disk 3 from the rotary feeder 2 to a tablet supply part 11. Upon reaching the tablet supply part 11, the tablet 51 is sucked to the first conveyance disk 4 and conveyed to a tablet delivery part 12. At the tablet delivery part 12, the tablet 51 is transferred from the first conveyance disk 4 to the second conveyance disk 5. On the second conveyance disk 5, the tablet 51 is conveyed in a standing state (upright posture) where the side surface 52 is directed in the vertical direction. First, the API is ejected/supplied from the medicinal agent supply device 6 to the tablet 51 on the second conveyance disk 5, and the API is applied into the hole 53. Thereafter, the tablet 51 is conveyed to the inkjet head 7 while being sucked to the second conveyance disk 5, and printing is applied to the front and back surfaces 54a and 54b by the inkjet head 7. Then, only non-defective products are discharged outside the device through a non-defective discharge part 13.

As illustrated in FIG. 1, the tablet 51 is supplied to the rotary feeder 2 from a not-shown hopper through a tablet supply path 14. The rotary feeder 2 is a so-called vibrationless rotary parts feeder and has a configuration in which a rotary disk 16 and an annular rotary plate 17 are coaxially provided in a cylindrical casing 15. The tablet 51 is supplied from the tablet supply path 14 onto the rotary disk 16 being rotated, moved in the circumferential direction with rotation of the rotary disk 16, and then moved to the annular rotary plate 17 side. The tablet 51 on the annular rotary plate 17 is moved in the circumferential direction with rotation of the annular rotary plate 17 to be fed to a tablet acquisition part 18. The tablet 51 fed to the tablet acquisition part 18 is sucked to one of suction holes (suction parts) 19 formed in an end face 3a of the tablet supply disk 3 being rotated. The suction holes 19 are connected to a suction device (not illustrated) such as a vacuum pump. On the tablet supply disk 3, the tablet 51 is sucked to the end face 3a in a horizontal posture (state where the front and back surfaces 54a and 54b face upward and downward or vice versa, respectively, in the vertical direction).

The tablet 51 sucked to the tablet supply disk 3 is fed to the tablet supply part 11 with rotation of the tablet supply disk 3 while maintaining the horizontal posture. Upon reaching the tablet supply part 11, the tablet 51 is transferred from the tablet supply disk 3 to the first conveyance disk 4. Circular suction holes (suction parts) 21 are formed in an end face 4a of the first conveyance disk 4 so as to be arranged in the circumferential direction. The suction holes 21 are also connected to a suction device such as a vacuum pump. The tablet 51 fed to the tablet supply part 11 is sucked to the end face 4a of the first conveyance disk 4 by one of the suction holes 21. At this time, the tablet 51 is held to the end face 4a in a state where one of the front and back surfaces 54a and 54b is sucked thereto.

A side surface inspection device 22 is disposed near the first conveyance disk 4. The side surface inspection device 22 is used for inspecting a state (presence/absence of cracking and chipping, positions of the holes 53) of the side surface 52 of the tablet 51 sucked to and supported by the first conveyance disk 4 (side surface inspection). The side surface inspection device 22 is provided with a camera, and an image photographed by the camera is sent to a not-shown controller, where determination of nondefective/defective is made. A light and a pair of prisms are provided in the photographing range of the camera. The pair of prisms are disposed so as to face the tablet side surface. The camera photographs the appearance of the tablet side surface illuminated by the light through the two prisms. The side surface inspection device 22 can not only inspect the outer appearance of the tablet 51, but also measure the thickness thereof. That is, the side surface inspection device 22 can also determine the presence/absence of a dimensional defect. A tablet 51 in which abnormality is detected is recognized as a defective and discharged through a defective discharge part 23 provided at the rear stage of the second conveyance disk 5 without being subjected to printing.

The second conveyance disk 5 is provided at the rear stage of the first conveyance disk 4. Like the first conveyance disk 4, the second conveyance disk 5 has in an end face 5a thereof circular suction holes (suction parts) 24 connected to a suction device and arranged in the circumferential direction. The end faces 4a and 5a of the respective first and second conveyance disks 4 and 5 are disposed so as to adjacently face each other at the tablet delivery part 12 in an orthogonal state. Upon reaching the tablet delivery part 12, the tablet 51 is sucked to one of the suction holes 24 formed in the end face 5a of the second conveyance disk 5 and conveyed to the second conveyance disk 5 side. The tablet 51 is sucked to the first conveyance disk 4 at its front or back surface 54a or 54b, so that at the tablet delivery part 12, the side surface 52 of the tablet 51 faces the end face 5a of the second conveyance disk 5. Accordingly, on the second conveyance disk 5 side, the side surface 52 of the tablet 51 is sucked, and the tablet 51 is held to the end face 5a in a standing state.

The medicinal agent supply device 6 is disposed at the rear stage of the tablet delivery part 12. The medicinal agent supply device 6 is provided with a nozzle 25 connected to a not-shown medicinal agent storage part. The medicinal agent storage part stores therein a predetermined API, the other tablet constituent components (various medicinal agents such as an excipient, an absorption promoter, a gel former, and a disintegrator), and a binder component etc. (hereinafter, they are collectively referred to as “API and the like”). In the medicinal agent storage part, the API and the like are melted or suspended in an adequate solvent according to a prescription. The API and the like are appropriately supplied by pressurization from the medicinal agent storage part to the nozzle 25 by a not-shown pump. The medicinal agent supply device 6 can continuously supply a constant amount of the API and the like. From the nozzle 25, a constant amount of droplets of the API and the like in a liquid or semi-solid state are ejected to the tablet 51 on the second conveyance disk 5 and supplied into the holes 53. The API and the like supplied into the holes 53 get dry and solidify to be retained in the tablet 51.

In the tablet production device 1, it is possible to produce various types of tablets on a single device by changing the type of the API to be supplied. In this case, only by changing the type of the API to be supplied from the medicinal agent supply device 6, different types of tablets can be produced. Thus, a significant facility revision of the like is not required for production of various types of tablets, making it possible to reduce device cost such as equipment investment cost.

Further, since the plurality of holes 53 are formed in the tablet 51, it is possible to separately incorporate, in the tablet 51, a plurality of APIs or a plurality of the other tablet constituent components (various medicinal agents such as an excipient, an absorption promoter, a gel former, and a disintegrator). In this case, the holes 53 are formed independently of each other, so that a combination of APIs in which degeneration is likely to occur due to blending or a combination of the API and medicinal agent such as an excipient can be added. This allows APIs for a specific disease to be incorporated in one tablet, which in turn allows production of a tailor-made tablet for each individual patient. Further, when an active ingredient required can be incorporated in one tablet, it is sufficient for a patient to take the one tablet instead of a plurality of tablets, whereby medication burden can be reduced.

Specifically, for example, assume that there is a patient who currently takes three tablets (three different types) in the morning, one tablet (one type) in the daytime, and five tablets (five different types) in the night. In this case, it is necessary for the patient to only take one tablet incorporating three different types of APIs in the morning, one tablet in the day time, and one tablet incorporating five different types of APIs in the night, thereby reducing medication burden. Further, reduction in the number of tablets reduces transportation cost.

A print surface inspection device 26 is disposed at the rear stage of the medicinal agent supply device 6 in proximity to the second conveyance disk 5. The print surface inspection device 26 inspects states of the front and back surfaces 54a and 54b of the tablet 51 sucked to and supported by the second conveyance disk 5 (print surface inspection). The print surface inspection device 26 is also provided with a camera, and an image photographed by the camera is sent to the not-shown controller, where determination of nondefective/defective is made. In the case of a scored tablet 51, the position of the scoring line is also detected by the print surface inspection device 26. As in the case of the side surface inspection device 22, a tablet 51 in which defective outer appearance is detected is recognized as a defective and discharged through the defective discharge part 23 without being subjected to printing.

The inkjet head 7 is provided at the rear stage of the print surface inspection device 26. The inkjet head 7 applies predetermined print processing to the front and back surfaces 54a and 54b of the tablet 51. At this time, the tablet 51 is supported with the side surface 52 sucked, and thus the front and back surfaces 54a and 54b are each completely exposed. Thus, in the tablet production device 1, printing can be applied to the entire surface of the tablet 51, so that the tablet 51 has no area where printing cannot be applied. Further, printing can be applied to the front and back surfaces 54a and 54b at the same time. Thus, the direction of characters printed on the front surface 54a of the tablet 51 and the direction of characters printed on the back surface 54b thereof can be made to coincide with each other according to the direction of the scoring line detected by the print surface inspection device 26.

As described above, the tablet 51 can be subjected to inspection or printing in a state where a printed surface is completely exposed, and thus there occurs no area where inspection or printing cannot be applied around the tablet. Further, in the tablet production device 1, both the front and back surfaces of the tablet 51 can be subjected to printing without involving reversal of the tablet 51, thereby eliminating a risk of ink transfer or print blurring associated with the reversal of the tablet. Further, the both surfaces of the tablet 51 can be subjected to printing at the same time, so that the directions of characters printed on the front and back surfaces 54a and 54b can be made to coincide with each other.

Further, in the tablet production device 1, the holes 53 are formed in the side surface 52 of the tablet 51, so that printing can be started immediately after application of the API and the like without waiting until the API and the like get dry. Thus, productivity of the tablet can be improved. Further, the API does not adhere to the front and back surfaces of the tablet produced by the tablet production device 1 and is stored and retained in the holes 53. This makes it difficult for the API to contact a container or another tablet during conveyance, thereby preventing the API part from peeling off.

In addition, since printing is performed along with addition of the API and the like, each individual tablet can be printed for a display associated with added API and the like, production date/time, or production site. Therefore, production conditions of the tablet can be recorded as a history and traced, allowing traceability to be ensured. Further, by printing the order of dosing, a patient's name, a dosing date/time, or a barcode or a two-dimensional code showing them on the tablet itself, medication management can be facilitated. Further, a patient's favorite color or mark can be designated and printed, whereby visibility can be further enhanced. Further, by performing printing in association with past medication troubles due to allergy etc. or DNA data, advanced medication management can be achieved. Furthermore, when the tablets are packaged in PTP in the order of dosing, medication management can be further facilitated. By adding the API to a placebo tablet as an orally disintegrating tablet, dosing itself can be facilitated.

A print inspection device 27 for checking a print result is provided at the rear stage of the inkjet head 7. The print inspection device 27 is also provided with a camera, and an image photographed by the camera is sent to the not-shown controller. When a printing defect is detected by the controller, the corresponding tablet is discharged through the defective discharge part 23 at the rear stage of the print inspection device 27. The defective discharge part 23 is provided with a discharger 28 for removing the defective from the second conveyance disk 5. The discharger 28 is formed into a gear-like shape and has a plurality of radially extending engagement projections 29. When a tablet 51 determined to have a defect in outer appearance or printing reaches the defective discharge part 23, the discharger 28 is rotated, and the defective tablet is dropped from the second conveyance disk 5 by the engagement projection 29 to be removed. On the other hand, a tablet 51 determined to be non-defective is discharged through the non-defective discharge part 13.

### Second Embodiment

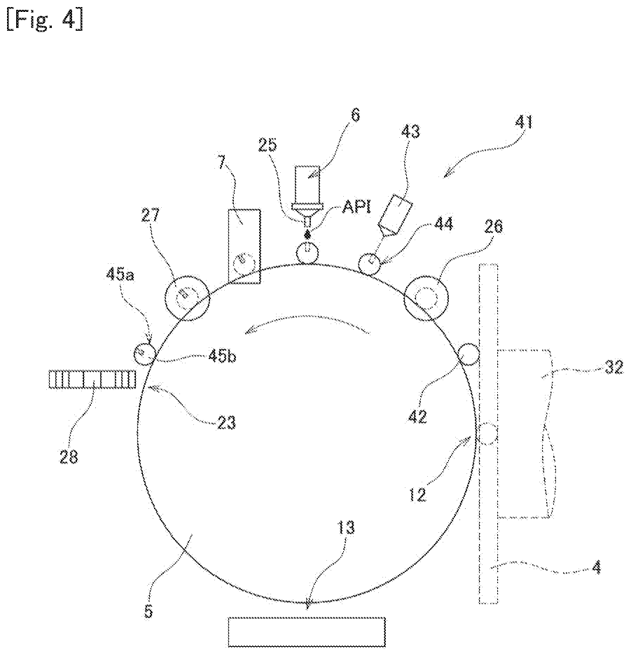

The following describes a tablet production device 41 according to a second embodiment of the present invention. FIG. 4 is an explanatory view illustrating the configuration of the tablet production device 41. Like the tablet production device 1 according to the first embodiment, the tablet production device 41 has a three-disk configuration. Although only the second conveyance disk 5 and its surrounding parts are illustrated in FIG. 4, other configurations are the same as those illustrated in FIG. 1. In the present embodiment, the same reference numerals are given to the same components as in the first embodiment and the description thereof will be omitted. The tablet production method according to the present invention can also be performed in the tablet production device 41, and the tablet 51 according to the present invention can be produced in the tablet production device 41.

In the tablet production device 41, the holes 53 are formed in the side surface of a tablet 42 having no holes on the device, and then addition of the API and printing are performed on the same device. In the tablet production device 1 according to the first embodiment, supply of the API into the holes 53 in the tablet side surface and printing on the front and back surfaces 54a and 54b are performed on a single device, while in the tablet production device 41, the holes 53 are also formed on the same device. To this end, the tablet production device 41 is further provided with a laser drilling machine (drilling unit) 43 for forming the holes 53. The laser drilling machine 43 may be a commercially available laser marker and is configured to irradiate the tablet side surface with a laser beam to thereby form the holes 53 in the radial direction.

The laser drilling machine 43 is provided at the front stage of the medicinal agent supply device 6 in proximity to the second conveyance disk 5. As illustrated in FIG. 4, the print surface inspection device 26, laser drilling machine 43, medicinal agent supply device 6, inkjet head 7, print inspection device 27, and discharger 28 are provided around the second conveyance disk 5 in this order from the first conveyance disk 4 side. Also in the tablet production device 41, the tablet 42 is transferred at the tablet delivery part 12 from the first conveyance disk 4 to the second conveyance disk 5. On the second conveyance disk 5, the tablet 42 is conveyed in a standing state where a side surface 44 is directed in the vertical direction.

The tablet 42 sucked to and supported by the second conveyance disk 5 is first subjected to inspection of the state of the front and back surfaces 45a and 45b by the print surface inspection device 26 (print surface inspection). Then, the holes 53 are formed in the side surface 44 by the laser drilling machine 43. After formation of the holes 53 in the tablet side surface, printing is applied to the front and back surfaces 45a and 45b by the inkjet head 7. Thereafter, the API is ejected/supplied from the medicinal agent supply device 6 and is applied into the hole 53. After the API application, a print result is checked by the print inspection device 27. When a printing defect is detected, the corresponding tablet is discharged through the defective discharge part 23, and only non-defective products are discharged outside the device through the non-defective discharge part 13.

As described above, in the tablet production device 41, the hole 53 are formed in the tablet side surface by the laser drilling machine 43, so that even when the tablet 42 having no holes in the side surface is used, API application and printing can be achieved on a single device. Thus, the application target of the tablet production method according to the present invention can be expanded, and the tablet according to the present invention can be produced more easily. Although printing is applied to the front and rear surfaces of the tablet by using the inkjet head 7 in the tablet production device 41 according to the second embodiment, printing or engraving may be applied by using the laser marker.

The present invention is not limited to the above embodiments, and various modifications may be made within the scope of the present invention.

For example, although a placebo tablet is used as the tablet 51 in the above embodiments, the API and the like can be applied to a tablet containing some sort of medicinal ingredient by forming the holes 53 in the tablet. Further, although the inkjet head 7 is provided at the rear stage of the medicinal agent supply device 6 in the above tablet production device 1, the positional relationship between the medicinal agent supply device 6 and the inkjet head 7 is arbitrary, and the inkjet head 7 may be provided at the front stage of the medicinal agent supply device 6. Similarly, the positional relationship between the inkjet head 7 and the medicinal agent supply device 6 in the tablet production device 41 is arbitrary. Further, more than one medicinal agent supply device 6 may be provided so as to allow plurality of kinds of APIs to be added. Further, a cartridge type medicinal agent supply device may be used so as to allow easy replacement of the API.

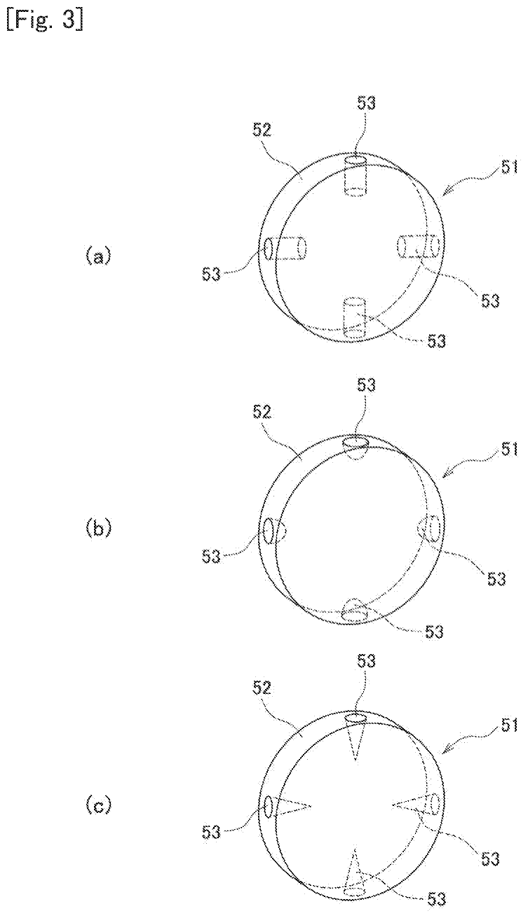

In addition, the holes 53 of the tablet 51 may not necessarily extend up to substantially the center of the tablet as illustrated in FIG. 2. For example, as illustrated in FIG. 3(a), the holes 53 may be formed shallow. Further, the size of the holes does not matter and may be minute. Further, the shape of the holes 53 may be a semi-spherical shape (FIG. 3(b)), a conical shape (FIG. 3(c)), or a square-hole shape, in addition to the columnar shape. Further, there are various modes for retaining the API and the like in the holes 53, including a mode in which the API and the like are filled in the hole and solidified and a mode in which the inner surface of the hole is coated with the API and the like.

Further, although circular tablets are used as a processing target of the tablet production device 1 in the above embodiments, the tablet production device according to the present invention may be applied not only to the circular tablets, but also to various types of tablets such as oblong tablets, caplets, and polygon-shaped tablets. Further, the inkjet head 7 includes an inkjet head 7a for front surface and an inkjet head 7b for back surface which are disposed so as to face each other at symmetrical positions; however, they may be displaced from each other. Further, a plurality of heads may be provided for multicolor printing. Further, in the tablet production device 1, printing is performed as well as adding the API and the like; however, printing is not necessary for an engraved tablet, so that the inkjet head 7 may not necessarily be provided in the device.

### INDUSTRIAL APPLICABILITY

The present invention may be applied not only to printing for medical tablets, but also to food products such as confectionary having a tablet shape. In this case, in place of the API, various flavor materials are added to the tablet-shaped confectionary, whereby a confectionary having various flavors can be produced in a single device.

1: Tablet production device

2: Rotary feeder

3: Tablet supply disk (conveyance unit)

3a: End face

4: First conveyance disk (conveyance unit)

4a: End face

5: Second conveyance disk (conveyance unit)

5a: End face

6: Medicinal agent supply device (supply unit)

7: Inkjet head (print unit)

7a: Inkjet head for front surface

7b: Inkjet head for back surface

11: Tablet supply part

12: Tablet delivery part

13: non-defective discharge part

14: Tablet supply path

15: Casing

16: Rotary disk

17: Annular rotary plate

18: Tablet acquisition part

19: Suction hole (suction part)

21: Suction hole (suction part)

22: Side surface inspection device

23: Defective discharge part

24: Suction hole (suction part)

25: Nozzle

26: Print surface inspection device

27: Print inspection device

28: Discharger

29: Engagement projection

31: Rotary shaft

32: Rotary shaft

33: Rotary shaft

41: Tablet production device

42: Tablet

43: Laser drilling machine (drilling unit)

44: Side surface

45a: Front surface

45b: Back surface

51: Tablet

52: Side surface

53: Hole (concave)

54a: Front surface

54b: Back surface

1. A tablet production device comprising a conveyance unit that conveys a tablet while sucking/supporting the tablet, wherein
the tablet has a concave in its side surface, and
the tablet production device has a supply unit that can add predetermined droplets to the concave of the tablet being conveyed by the conveyance unit.

2. The tablet production device according to claim 1, wherein
the concave is a hole opened to the side surface and extending in a radial direction of the tablet.

3. The tablet production device according to claim 2, wherein
a plurality of the concaves are formed in the circumferential direction of the tablet.

4. The tablet production device according to claim 1, wherein
the droplets contain a predetermined active pharmaceutical ingredient.

5. The tablet production device according to claim 1, further comprising a print unit provided in proximity to the conveyance unit and configured to apply printing to at least one of the front and back surfaces of the tablet being conveyed by the conveyance unit.

6. The tablet production device according to claim 1, wherein
the conveyance unit sucks/supports the side surface of the tablet such that the front and back surfaces of the tablet are each fully exposed.

7. The tablet production device according to claim 1, wherein
the conveyance unit is a conveyance disk formed in a disk shape, and
the conveyance disk has suction parts arranged in an end face thereof along the circumferential direction and configured to suck the side surface of the tablet.

8. A tablet production device comprising a conveyance unit that conveys a tablet while sucking/supporting the tablet, wherein
the tablet production device has a drilling unit that forms a concave in a side surface of the tablet and a supply unit that can add predetermined droplets to the concave of the tablet being conveyed by the conveyance unit.

9. A tablet production method comprising:
conveying a tablet having a concave in its side surface while sucking/supporting the tablet, and
adding predetermined droplets to the concave of the tablet being conveyed.

10. The tablet production method according to claim 9, wherein
the droplets contain a predetermined active pharmaceutical ingredient.

11. The tablet production method according to claim 9, wherein
the tablet is conveyed with the side surface thereof sucked/supported, and
printing is applied to at least one of the front and back surfaces of the tablet being conveyed.

12. A tablet having a hole opened to its side surface and extending in a radial direction thereof, wherein
a plurality of the holes are formed in the circumferential direction of the tablet, and
a predetermined active pharmaceutical ingredient is retained in at least one of the holes.

13. The tablet according to claim 12, wherein different types of active pharmaceutical ingredients are retained in the holes.

14. A tablet traceability method wherein:
a tablet that has a hole opened to its side surface and extending in a radial direction thereof and in which a predetermined active pharmaceutical ingredient is retained is used, and
information corresponding to the active pharmaceutical ingredient is printed onto at least one of the front and back surfaces of the tablet so as to allow traceability to be ensured.

15. A tablet medication management method comprising:
a tablet that has a hole opened to its side surface and extending in a radial direction thereof and in which a predetermined active pharmaceutical ingredient is retained is used; and
information corresponding to the active pharmaceutical ingredient is printed onto at least one of the front and back surfaces of the tablet so as to allow tablet medication to be managed.
